(19) United States
(12) Patent Application Publication
(10) Pub. No.: US 2017/0306337 A1
(43) Pub. Date: Oct. 26, 2017

(54) BIOPRODUCT FORMATION FROM A PLASMID ADDICTION SYSTEM IN THE ABSENCE OF CO-INDUCERS AND ANTIBIOTICS

(71) Applicant: OHIO STATE INNOVATION FOUNDATION, Columbus, OH (US)

(72) Inventors: F. Robert TABITA, Dublin, OH (US); Rick Avalos LAGUNA, Columbus, OH (US); Sarah Jeanne YOUNG, Gahanna, OH (US)

(21) Appl. No.: 15/528,301

(22) PCT Filed: Nov. 19, 2015
(86) PCT No.: PCT/US15/61532

§ 371 Date: May 19, 2017

Related U.S. Application Data
(60) Provisional application No. 62/082,336, filed on Nov. 20, 2014.

(51) Int. Cl. C12N 15/70; C12P 7/16; C12N 9/14; C12N 9/02; C12N 9/04; C12N 15/52

(52) U.S. Cl. CPC C12N 15/70; C12Y 306/03; C12Y 103/05004; C12Y 101/01027; C12Y 101/01001; C12N 9/0006; C12N 15/52; C12N 9/14; C12N 9/001; C12P 7/16

(57) ABSTRACT

Described herein are plasmid addiction systems comprising a host cell comprising one or more inactivated host cell essential genes; and a plasmid comprising one or more plasmid essential genes operably linked to a constitutively active promoter. Also described herein are metabolism-based plasmid addiction systems (PAS) comprising a host cell and a plasmid operably linked to a constitutively active promoter for producing value-based products (e.g., 1-butanol) and methods of generating PASs in microorganisms and producing 1-butanol from a PAS in the absence of antibiotics and/or co-inducers.

### CROSS REFERENCE TO RELATED APPLICATIONS

This application claims the benefit of U.S. Provisional Application No. 62/082,336, filed Nov. 20, 2014, which is incorporated herein by reference in its entirety.

### STATEMENT REGARDING FEDERALLY FUNDED RESEARCH

This invention was made with government support under grant number DE-AR000009 awarded by U.S. Department of Energy (FRT and STY). The government has certain rights in the invention.

### BACKGROUND

The use of microorganisms as biological factories for the production of value-based products is becoming commonplace due to the increased metabolic knowledge of selected host microorganisms. Synthetic biology methods have led to the production of many value-based products, such as human insulin, proteases, and antibiotics. Through manipulation of native biochemical pathways combined with the use of synthetic biology principles, key genes encoding for desired foreign biochemical pathways can be used for the synthesis of these products. Industrial production of value-based products, however, is hampered by costs associated with the need to supplement large microbial cultures with expensive, but necessary, co-inducer compounds, and antibiotics that are required for up-regulating gene expression and maintaining plasmid-borne synthetic genes, respectively.

Large scale production of many biofuels (e.g., alcohol) is contaminated with antibiotics that are commonly added to processes (e.g., fermentation) to increase biofuel production. Overuse and contamination of materials with antibiotics can lead to drug-resistance bacteria that can flow across the microbial system. In the United States, the Centers for Disease Control reported that 23,000 Americans die each year from infections caused by drug-resistant bacteria. Thus, a need exists for cost-efficient alternative and robust systems and techniques for producing important biofuels and value-added products without the use of antibiotics.

### SUMMARY

Described herein are plasmid addiction systems comprising a host cell comprising one or more inactivated host cell essential genes; and a plasmid comprising one or more plasmid essential genes operably linked to a constitutively active promoter.

Described herein are methods of producing a recombinant cell, the method comprising (a) contacting a host cell comprising one or more inactivated host cell essential genes with a plasmid comprising one or more plasmid essential genes operably linked to a constitutively active promoter; (b) selecting the recombinant cell comprising said plasmid.

Described herein are methods of producing 1-butanol, the method comprising (a) contacting a host cell comprising one or more inactivated host cell essential genes with a plasmid comprising one or more plasmid essential genes operably linked to a constitutively active promoter; (b) incorporating the plasmid into the host cell; (c) culturing the host cell under anaerobic conditions in the absence of a co-inducer and/or antibiotic; (d) producing a stable recombinant cell line; and (e) fermenting the recombinant cell line in nutrient medium under conditions suitable for the production of 1-butanol, thereby producing 1-butanol.

### BRIEF DESCRIPTION OF THE DRAWINGS

FIG. 1 illustrates the conceptual model of a plasmid addiction system (PAS) and production of butanol by various strains. (FIG. 1A) The lptB gene plasmid addicted 1-butanol production system. The chromosomal lptB gene was inactivated and placed within the synthetic plasmid-based 1-butanol operon to construct plasmid p91BuOH(1)lptB. The constitutive cbbL gene promoter was used to drive expression of the essential synthetic operon. In this system, the requirement of co-inducer to drive gene expression was negated and the use of an antibiotic was not required for plasmid stability. (FIG. 1B) Small-scale semi-aerobic 1-butanol production in plasmid addicted strains. Strains RKE01, RKE03 (ΔadhE), RKE05 (ΔadhE, Δpta), RKE07 (ΔadhE, Δpta, ΔldhA), and RKE09 (ΔadhE, Δpta, ΔldhA, ΔfrdABCD) were cultured and 1-butanol levels monitored as described in Example 1.

FIG. 2 shows the fermentation kinetics of strain RKE09 and stability after multiple transfers. (FIG. 2A) Fermentation kinetics of RKE09 under aerobic growth conditions with pH controlled at 7.0 with 5 N NaOH. Aerobic growth consisted of a culture volume of 150 ml in a 250 ml flask. Gas chromatography was used to measure the levels of 1-butanol, ethanol, acetate, and butyrate. High performance liquid chromatography was used to measure glycerol levels. (FIG. 2B) 1-butanol production by strain RKE09 grown under aerobic semi-continuous batch culture conditions. Batch cultures were grown for 72 h and 1-butanol production was measured at the end of each batch culture cycle. The preceding batch culture served as the inoculum for the ensuing batch culture.

### DETAILED DESCRIPTION

The present invention can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., an animal. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

### Plasmid Addiction System

Described herein are plasmid addiction systems comprising a host cell comprising one or more inactivated host cell essential genes; and a plasmid comprising one or more plasmid essential genes operably linked to a constitutively active promoter.

Described herein are metabolism-based plasmid addiction systems in Escherichia coli for the production of a model bioproduct, 1-butanol, which retains the benefits of a plasmid-based system but without the cost associated with co-inducers to up-regulate gene expression or antibiotics to maintain plasmid stability. In some aspects, the disclosed plasmid addiction systems abrogate environmental issues associated with large-scale usage of antibiotics.

Described herein are metabolism-based plasmid addiction systems (PAS) in a model microorganism, such as Escherichia coli (E. coli) that relies on the expression of one or more essential genes and utilizes an active constitutive promoter. The PASs described herein are also capable of producing value-based products or bioproducts. The systems described herein abrogate the need for expensive antibiotics and co-inducer molecules so that plasmid-borne synthetic genes can be expressed at high levels in a cost-effective manner. Accordingly, in some aspects, the methods and systems described herein features a plasmid addiction system comprising a host cell comprising one or more inactivated host cell essential genes and a plasmid comprising one or more plasmid essential genes operably linked to a constitutively active promoter. The PAS systems described herein can be applied to other bacteria where the constitutive promoter is active and where convenient essential genes may be readily inactivated. For example, the PAS systems described can be applied to other bacteria such as the carbon dioxide assimilating hydrogen bacterium Ralstonia eutropha.

Plasmid addiction systems are classified based on their function and are divided into three major groups: toxin/antitoxin-based systems, operator repressor titrations systems and metabolism-based systems. Metabolism-based addiction systems can be further divided into two groups: catabolism-and anabolism-based systems. The present invention discloses a synthetically engineered catabolism-based PAS. In an acatabolism-based PAS, one or more genes are required or essential for a catabolic pathway (e.g., a metabolic pathway). Metabolic pathways (e.g., fermentation in bacteria) are comprised of multi-step modifications of a starting molecule or substrate to form another product or molecule. Changes in the availability and/or concentrations of any intermediate product can influence the rate of production of the end product. Knowledge of a catabolic pathway is important to the development of a catabolism-based PAS. For example, if a gene is inactivated in a host cell and a copy of the intact gene is transferred or localized in a plasmid, the production of that particular product or molecule will now rely on or become dependent upon the existence of the plasmid. In the present invention, a metabolism-based PAS relies on the expression of one or more essential genes and utilizes a constitutively active promoter to drive gene expression in the plasmid. This concept can be analogously applied to produce stable value-based products or bioproducts (e.g., biofuel, including butanol and solvents such as acetone, alcohol, butanol) in large quantities in the absence of expensive antibiotics and co-inducer molecules.

Although synthetic biology approaches have led to improvements in the bioengineering microorganisms to produce human insulin, proteases, antibiotics, and biofuels, plasmid-free cells are associated with a loss in the value-based product recovery and thus, profitability. The metabolism-based plasmid addiction system of the present invention relies on the strict natural selection of plasmid-containing cells due to the expression of a plasmid-encoded gene(s) that is required for the viability of the bacterium. Therefore, cells that maintain a plasmid containing the essential gene(s) and a suite of value-based product gene(s) are viable and able to produce the desired product. To date, all recently employed plasmid addiction systems (Voss, I. and Steinbuchel, A., Metab. Eng. 8, 66-78 (2006); Kroll, J., Klinter, S. and Steinbuchel, A., Appl. Microbiol. Biotechnol. 89, 593-604 (2011); Shen, C. R. et al., Appl. Environ. Microbiol. 77, 2905-2915 (2011); and Wong, M. S. et al., Appl. Environ. Microbiol. 80, 3276-3282 (2014)) negate the requirement of antibiotics for plasmid stability. Each of these PASs, however, relies on specific constraints, for example, the use of a specific carbon source or medium-specific growth condition, as well as the need for co-inducer supplementation for up-regulated gene expression. These constraints limit the flexibility for industrial scale production.

The plasmid addiction strategy disclosed herein, involves the expression of the essential plasmid-borne gene, which is co-expressed with genes of a synthetic 1-butanol pathway, thus ensuring that microbial growth and cell viability involves synthesis of the desired product (e.g., 1-butanol), without the need for antibiotics to maintain plasmid stability. In addition, when anaerobic growth was employed, the production of 1-butanol served as a second essential system for cell viability, thus increasing the stringency for plasmid addiction. One advantage of the plasmid addiction system of the present invention is that it employs a promoter that permits constitutive expression of desired genes, so that expensive cofactors such as isopropyl-B-D-galactopyranoside (IPTG) are unnecessary. Although the synthesis of 1-butanol was used as a model system, the plasmid addiction strategy as described herein can be easily adapted to the production of other industrially significant products, for example, solvents, such as acetone and alcohol and biofuel such as ethanol.

The term “essential genes” as used herein refers to those genes that are required for survival, viability and/or growth of a particular cell or organism. The essential gene or genes can be different depending on the organism. We use the term “host cell essential genes” to mean the gene or genes that are required for survival, viability and/or growth in a host cell (e.g., E. coli). For example, “essential genes” can be genes that are responsible for the expression of compound (e.g. proteins or peptides) that are required for survival, viability and/or growth of a particular cell or organism. An example of a compound required for survival and viability in E. coli is lipopolysaccharide whose anabolic synthesis is absolutely required for cell viability. The gene or genes that are essential host cell are also endogenous to the host cell. Other catabolic systems that can be targeted for inactivation include, but are not limited to, fermentation. For example, inactivation of the essential genes that encode alcohol dehydrogenase, lactate dehydrogenase, and fumarate reductase enzymes prevents anaerobic growth of E. coli.

The term “inactive,” “inactivated” or “silenced” refers generally to a gene no longer controlling the production of a particular protein or substrate or molecule. The expression of the gene, for example, can be inactivated (complete or partial). In some instances, expression of the gene is reduced, and not necessarily eliminated; while in other instances, the gene can be completely removed from the organism's genome resulting in a “knockout.”

As described herein, the PAS can be further defined by the host cell essential genes that are inactivated including but not limited to one or more genes that are required for cell viability, survival and/or growth. Thus, in some aspects, the invention features host cell essential genes that can be inactivated including one or more genes associated with a metabolic pathway of interest (e.g., a fermentative pathway). In some aspects, an entire metabolic pathway can be inactivated in the host cell. In some aspects, one or more inactivated host cell essential genes can result in or lead to an imbalance in the redox balance of the host cell, thereby having a negative effect on cell viability, survival and/or growth. In some aspects, the invention features a PAS wherein one or more inactivated host cell essential genes negatively effects and/or imbalances the redox balance of the host cell.

The inactivated host cell essential genes can also encode one or more genes required for cell survival, growth and/or viability. In one aspect, one or more of the inactivated host cell essential genes are associated with lipopolysaccharide biogenesis (e.g., lptB). In some aspects, the inactivated host cell essential genes can include one or more genes required for cell survival, growth and/or viability along with one or more genes associated with metabolic pathway.

The PAS can also be further defined by the plasmid and the plasmid essential genes in the host cell, as described further below. The plasmid need not be integrated into the genome of the host cell, but rather needs only to be incorporated and maintained by the host cell. Once the plasmid is incorporated into and maintained by the host cell, the plasmid essential genes can then expressed. In some aspects, the expression of one or more plasmid essential genes in the host cell restores the redox balance or redox state of the host cell. In some aspects, the host cell essential genes and the plasmid essential genes can be the same and/or different. In some aspects, the expression of one or more plasmid essential genes in the host cell leads to a balanced redox state (i.e., restores the redox state or balance of the host cell) and/or can rescue (i.e., compensate for the inactivated host cell genes) the host cell from cell death.

The term “plasmid” or “construct” refers to a small, circular nucleic acid sequence capable of transporting into a cell (e.g., a host cell) another nucleic acid or one or more genes to which the plasmid DNA sequence is linked. Plasmids of the present invention can be naturally occurring or constructed. Most plasmids do not encode information required for cell viability or survival and can be used to introduce the formation of specific proteins or even establish entire metabolic pathways.

The term “plasmid essential genes” refers to a gene or genes that are present in a plasmid. These genes can be exogenous genes that are operably linked to a control element, such as a promoter. Methods to transfer a gene or genes to a plasmid are well known in the art. The plasmid essential genes of the present invention can be the same genes that were inactivated in the host cell and/or can be different. The plasmid essential genes need to be capable of enabling the host cell to remain viable, survive and/or grow. In some instances, the same chromosomal gene (e.g., lptB, the gene responsible for the production of lipopolysaccharide) that was inactivated in the host cell is placed within the synthetic plasmid. In other instances, when more than one gene is inactivated, for host cell survival, it can be necessary to maintain a plasmid that expresses a set of genes (e.g., a synthetic 1-butanol operon) in addition to an essential gene. In other words, plasmids can be used to introduce the formation of additional proteins and/or establish a complete metabolic pathway. Collectively, the essential gene and the expression of a set of genes present in a synthetic plasmid required for host cell viability, survival and/or grow are referred to as “plasmid essential genes.” Plasmid essential genes can be or include genes of interest. Further, in some instances, one or more of the plasmid essential genes can rescue the growth of the host cell by restoring, for example, the redox balance of the host cell that was lost due to the inactivation of one or more of the host cell essential genes. The constitutively active promoter can be operably linked to the so-called “rescue” gene(s).

The plasmid essential genes described herein can encode one or more genes associated with fermentation (e.g., alcohol dehydrogenase, lactate dehydrogenase and/or fumarate reductase). In some aspects, the plasmid essential gene comprises a synthetic 1-butanol operon. Accordingly, the systems and methods described herein also features a PAS capable of producing 1-butanol. In some aspects, the systems and methods described herein features a PAS wherein the inactivated host cell essential gene is lptB and the plasmid essential gene comprises lptB and a synthetic 1-butanol operon or the lptB is co-expressed with one or more genes of a synthetic 1-butanol pathway. In some aspects, the plasmid essential genes can be one or more genes of interest.

The redox system maintains homeostasis in a cell. Redox reactions include all chemical reactions in which an atom's oxidation state changes and involves the transfer of one or more electrons. Oxidation refers to the loss of electrons by a molecule, atom or ion while reduction refers to the gain of electrons by a molecule, atom or ion. Cells maintain redox balance by producing or eliminating a reactive oxygen and/or nitrogen species (ROS/RNS). Examples of reactive oxygen species include superoxide (O₂⁻), hydroxyl radical (HO) and hydrogen peroxide (H₂O₂). Examples of reactive nitrogen species include nitric oxide (NO) and peroxynitrite (ONOO⁻). Endogenous sources can contribute to the formation of intracellular ROS/RNS and thereby resulting in an imbalanced redox sate. Examples of endogenous sources of ROS include, but are not limited, NAD(P)H oxidase, cytochrome c oxidase, and xanthine oxidase. The term “redox state” describes the balance of a redox reaction in a cell (e.g., NAD⁺/NADH and NADP⁺/NADPH). Because the redox system of a cell is critical for homeostasis, an imbalanced redox state can induce cell death.

It is within the scope of the present disclosure to target genes or inactivate one or more genes in the host cell that can result in an imbalanced redox state including but not limited to genes that encode for the production of formate dehydrogenase and/or any of the proteins associated with the pyruvate dehydrogenase complex (e.g., pyruvate dehydrogenase, dihydrolipoly transacetylase and dihydrolipoyl dehydrogenase. Likewise, the plasmid essential genes can include one or more of the genes that are inactivated in the host cell.

We use the term “compensate” to mean to provide a counterbalance or to be equivalent or to produce equilibrium to what is lacking, absent, not found, silenced, inactive, inactivated or knocked out. For example, one or more essential genes present in a plasmid (e.g., plasmid essential gene(s)) can compensate for the lack of cell growth, viability and/or survival if one or more essential genes in a host cell (e.g., host cell essential gene(s)) are inactivated.

The phrase “gene of interest” or “genes of interest” can mean a nucleic acid sequence (e.g., an essential gene), that is partly or entirely heterologous, i.e., foreign, to a cell into which it is introduced. “Gene of interest” can also mean a nucleic acid sequence that is partly or entirely homologous to an endogenous gene of the cell into which it is introduced, but which is designed to be inserted, for example, into a plasmid in such a way as to drive expression of the gene or genes of interest in a host cell. For example, a gene(s) of interest can be one or more that genes associated with a metabolic pathway (e.g., fermentation) or cell survival (e.g., lptB). Typically, the gene of interest is one or more genes that are inserted into a plasmid and can be the same gene or genes that were inactivated in a host cell or at least is not present or fully expressed in a host cell.

The term “operatively linked to” refers to the functional relationship of a nucleic acid (or gene or genes or gene of interest) with another nucleic acid sequence (or one or more genes). Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA. Accordingly, in an aspect, one or more of the plasmid essential genes are operably linked to a control element, such as a promoter. The promoter is constitutively active or inducibly active. Typically, the promoter is cbbL is from R. eutropha.

Methods of silencing, inactivating or knocking out genes is well known in the art. For example, short interfering RNAs (siRNAs), also known as small interfering RNAs, are double-stranded RNAs that can induce sequence-specific post-transcriptional gene silencing, thereby decreasing gene expression. siRNAs can be of various lengths as long as they maintain their function. In some examples, siRNA molecules are about 19-23 nucleotides in length, such as at least 21 nucleotides, and for example at least 23 nucleotides. siRNAs can effect the sequence-specific degradation of target mRNAs when base-paired with 3′ overhanging ends. The direction of dsRNA processing determines whether a sense or an antisense target RNA can be cleaved by the produced siRNA endonuclease complex. Thus, siRNAs can be used to modulate transcription or translation, for example, by decreasing expression of phaA, phaB1, phaC1, phaC2, or a combination thereof. SiRNAs can also be used to modulate transcription or translation of other genes of interest as well. (See, e.g., Invitrogen's BLOCK-IT™ RNAi Designer).

In some aspects, the PAS comprises an active constitutive promoter. Described herein are plasmids comprising one or more plasmid essential genes operably linked to a promoter, for example, from Ralstonia eutropha (e.g., cbbL).

### Host Cells

In some aspects, the host cell of the PAS of the present invention is a grain negative bacteria. Examples of gram negative bacteria include, but are not limited to gram negative bacteria from the phylum Proteobacteria (Escherichia, Ralstonia, Rhodopseudomonas, Rhodobacter, Rhodospirillum). Additional types of gram negative bacteria that are suitable host cells include, but are not limited to, chemoautotrophic bacteria (e.g., hydrogen-oxidizing bacteria (sometimes referred to as “knallgas” bacteria) such as Ralstonia eutropha and other Ralstonia species as well as non-sulfur purple bacteria (also referred to as purple bacteria or purple photosynthetic bacteria; e.g., Rhodospirillum, Rhodopila, Rhodopseudomonas, Rhodomicrobium, Rhodobium, Rhodobacter, Rhodocyclus, Rhodoferax).

The PASs described herein can grow under aerobic, semi-aerobic (i.e., partially aerobic), anaerobic, and/or high cell density conditions. In some aspects, anaerobic growth conditions are preferred.

### Methods of Producing a Recombinant Cell, Recombinant Cell Line or Recombinant Strain

Described herein are methods of producing a recombinant cell, recombinant cell line or recombinant strain. The methods can include the steps of contacting a host cell (such as a gram negative bacteria, including but not limited to E. coli) comprising one or more inactivated host cell essential genes with a plasmid comprising one or more plasmid essential genes operably linked to a constitutively active promoter (e.g., from Ralstonnia eutropha, such as cbbL) and includes the step of selecting for a recombinant cell comprising said plasmid. The methods can further comprise the step of culturing the recombinant cell in anaerobic conditions in the absence of a co-inducer and/or antibiotic.

In some aspects, the recombinant cells or cell lines produced by the method disclosed herein can produce a value-based product (e.g., butanol, 1-butanol). Thus, the methods can further include the steps of fermenting the cell (or cell line) in a nutrient medium and recovering the value-based product. In some aspects, the recovered bioproduct is 1-butanol. The amount of the 1-butanol recovered can be from about 50 mg/L to about 2.3 g/L. The amount of recovered 1-butanol can be further increased (or decreased) by additional or even different manipulations to the strain. Such manipulations can be associated with the selection of one or more host cell essential genes targeted for inactivation and/or the subsequent selection of one or more plasmid essential genes incorporated into the plasmid along with the appropriate constitutively active promoter.

In some aspects, the methods of producing a recombinant cell features one or more inactivated host cell essential genes that result in an imbalance of the redox state or imbalances the redox balance of the host cell.

In some aspects, the methods of producing a recombinant cell features one or more plasmid essential genes that restore the redox balance of the host cell. The plasmid essential genes can comprise a synthetic 1-butanol operon and/or one or more genes that encode one or more genes associated with fermentation.

In some aspects, the invention features methods of producing 1-butanol. The methods can include the steps of contacting a host cell with a plasmid, wherein the host cell comprises one or more inactivated host cell essential genes and wherein the plasmid comprises one or more plasmid essential genes operably linked to a constitutively active promoter; incorporating the plasmid into the host cell; culturing the host cell under anaerobic conditions in the absence of a co-inducer or antibiotic; producing a stable recombinant cell line; fermenting the stable recombinant cell line in nutrient medium under conditions suitable for the production of 1-butanol and can include the step of producing 1-butanol. The methods described herein can also include the step of producing a product that generates or is capable of generating oxidized pyridine nucleotides or any other electron acceptors that are capable of balancing the redox potential or state of a cell. For example, the methods described herein can include the reductive pentose phosphate pathway to allow nonsulfur purple bacteria to balance the redox potential of the cell via the use of CO₂ as electron acceptor [Wang, X., Falcone, D. L., and Tabita, F. R. Reductive pentose phosphate-independent CO₂ fixation in Rhodobacter sphaeroides and evidence that ribulose bisphosphate carboxylase/oxygenase activity serves to maintain the redox balance of the cell. J. Bacteriol 175 (1993) 3372-3379.]

The methods described herein can further include one or more host cell essential genes that encode the proteins required for lipopolysaccharide biogenesis. The methods described herein can also include one or more plasmid essential genes required for host cell viability such as genes that encode the proteins required for lipopolysaccharide biogenesis and genes that encode one or more essential genes required for fermentation. The methods described herein can further comprise one or more plasmid essential genes that can rescue the growth of the stable recombinant cell line by restoring the redox balance of the host cell.

The PASs described herein can be based on the targeting of two metabolic pathways: fermentative pathway and lipopolysaccharide biogenesis. The PASs can comprise three components. For example the PASs described herein can comprise: (1) inactivation of one or genes associated with fermentation and the production of lipopolysaccharide; (2) the complementation by the plasmid-encoded artificial fermentative pathway mediated by 1-butanol operon and the essential lptB gene which allows the synthesis of lipopolysaccharide; and (3) a constitutively active promoter that drives the metabolic synthesis of 1-butanol. The PASs described herein represents a major potential source for producing and manufacturing value-based products such as biofuel. The use of a PAS as described herein can provide major economic and environmental advantages. For example, using the PASs described herein is an improvement of preexisting methods of producing butanol at a technical scale without the need for antibiotics and/or expensive co-inducers.

### Culture Conditions

Culture conditions of the present disclosure can include aerobic, anaerobic, semi-aerobic, or high cell density growth or maintenance conditions. Exemplary aerobic and anaerobic conditions have been described previously and are well known in the art. Any of these conditions can be employed with the bacteria of the present invention (e.g., E. coli) as well as other aerobic or anaerobic or high cell density conditions well known in the art. The culture conditions can include, for example, liquid culture procedures as well as fermentation and other large scale culture procedures. As described herein, yields of the value-based products of the invention, such as 1-butanol, can be obtained under aerobic, anaerobic, semi-aerobic or high density culture conditions.

An exemplary growth condition for producing 1-butanol, as disclosed herewith includes anaerobic culture, aerobic and/or fermentation conditions. In certain embodiments, the PAS comprising a host cell (e.g., E. coli) of the invention can be sustained, cultured, or fermented under anaerobic conditions. Briefly, anaerobic conditions refer to an environment in the absence of oxygen while aerobic conditions refer to an environment in the presence of oxygen.

The culture conditions described herein can be scaled up and grown continuously for manufacturing of 1-butanol. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of 1-butanol. Generally, and as with non-continuous culture procedures, the continuous and/or near-continuous production of 1-butanol will include culturing a non-naturally occurring 1-butanol producing organism of the invention in sufficient nutrients and medium to sustain and/or nearly sustain growth in an exponential phase. Continuous culture under such conditions can be included, for example, 1 day, 2, 3, 4, 5, 6 or 7 days or more. Additionally, continuous culture can include 1 week, 2, 3, 4 or 5 or more weeks and up to several months. Alternatively, the disclosed PAS of the invention can be cultured for hours, if suitable for a particular application. It is to be understood that the continuous and/or near-continuous culture conditions also can include all time intervals in between these exemplary periods. It is further understood that the time of culturing the PAS disclosed herein for a sufficient period of time to produce a sufficient amount of product for a desired purpose.

Typically, the growth medium that is useful for the producing 1-butanol is Terrific Broth comprising 12 g tryptone, 24 g yeast extract, 2.31 g KH₂PO₄, 12.54 g K₂HPO₄, per liter of water, supplemented with glycerol or glucose. Under certain conditions, such as anaerobic, glucose supplementation is preferred. Terrific broth is commercially available and can be modified by one skilled in the art.

Fermentation procedures are well known in the art. Briefly, fermentation for the biosynthetic production of 1-butanol can be utilized in, for example, fed-batch fermentation and batch separation; fed-batch fermentation and continuous separation, or continuous fermentation and continuous separation. Examples of batch and continuous fermentation procedures are well known in the art.

### EXAMPLES

The following examples are put forth so as to provide those of ordinary skill in the art with a complete disclosure and description of how the compounds, compositions, and methods claimed herein are used and evaluated and are intended to be purely exemplary of the invention and are not intended to limit the scope of what the inventors regard as their invention. However, those of skill in the art should, in light of the present disclosure, appreciate that many changes can be made in the specific aspects which are disclosed and still obtain a like or similar result without departing from the spirit and scope of the invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Development of a Plasmid Addicted System for 1-butanol Synthesis in Escherichia coli that is Independent of Co-Inducers, Antibiotics and Specific Carbon Source Additions

To develop a plasmid addicted 1-butanol production system in Escherichia coli that negates the need for expensive co-inducers and antibiotics, and is not limited by medium, carbon source, or growth condition, an anabolism-based plasmid addiction system for the production of 1-butanol, which relies on two essential metabolic pathways, was used. Lipopolysaccharide (LPS) biogenesis (anabolism), specifically the essential lptB gene, was the first essential anabolic system targeted. LPS is a cell surface glycolipid that is required for viability of E. coli. LptB is also essential for survival because it functions as the ATPase of the ABC transporter that powers LPS transport to the cell surface (Sperandeo, P. et al., J. Bacterial. 190, 4460-4469 (2008); Sherman, D. J. et al., Proc. Natl. Acad. Sci. U.S.A. 111, 4982-4987 (2014)). LPS biogenesis was targeted for plasmid addiction, as LPS synthesis is essential regardless of carbon source or growth condition (aerobic or anaerobic). Fermentation, specifically essential genes that encode alcohol dehydrogenase, lactate dehydrogenase, and fumarate reductase enzymes, was the second essential catabolic system targeted. Inactivation of these genes prevents anaerobic growth of E coli, however, 1-butanol production rescues growth by restoring redox balance (Shen, C. R. et al., Appl. Environ. Microbiol. 77, 2905-2915 (2011)). Therefore, this system establishes a stringent basis for maintaining plasmid stability by providing two essential plasmid-based metabolic systems under anaerobic growth conditions, which is a preferred growth condition for various industrial processes.

The following paragraphs describe the methods carried out.

Reagents. The chemicals used were acquired from Sigma-Aldrich (St. Louis, Mo.) or Fisher Scientifics (Pittsburgh, Pa.). Oligonucleotides were purchased from Sigma-Aldrich (St. Louis, Mo.) Phusion High-Fidelity DNA polymerase and restriction enzymes were obtained from New England Biolabs (Ipswich, Mass.). Invitrogen T4 ligase was obtained from Life Technologies (Grand Island, N.Y.).

Bacterial strains. E. coli strain JM109 (Yanisch-Perron, C., Vieira, J. and Messing, J., Gene 33, 103-119 (1985)) was used for propagation of plasmids and strain BW25113 (Datsenko, K. A. and Wanner, B. L., Proc. Natl. Acad. Sci. U.S.A. 97, 6640-6645 (2000)) was used for plasmid-addicted 1-butanol production.

Plasmid and strain construction. Plasmids and strains used in this study are listed in Table 1. To construct plasmids p62, p72, p91, p91BuOH(1), and p91BuOH(1)lptB, the multiple cloning site from pUC19, particularly the nucleotide sequence that spans the SacI and HindIII restriction sites, was subcloned into plasmid 3716 (a derivative of pLO11 (Schwarze, A., Kopczak, M. J., Rogner, M. and Lenz, O., Appl. Environ. Microbiol. 76, 2641-2651 (2010)) with a pBAD promoter and the araC gene), kindly provided by Dr. Oliver Lenz, Berlin, Germany), replacing the existing nucleotide sequence between the SacI and HindIII restriction sites. As a result, the number of available restriction sites for cloning purposes was expanded, resulting in the construction of plasmid p62. A NcoI restriction site was introduced within the araC gene of p62 by site directed mutagenesis, thus resulting in plasmid p72. This allows one to replace the existing pBAD promoter with a promoter of choice flanked by NcoI and SacI restriction sites. The promoter sequence of the chromosomal cbbL gene from R. eutropha strain H16 was amplified from genomic DNA and cloned into pUC19. Site-directed mutagenesis was then performed on the cbbL, promoter region. Specifically, the nucleotide sequence of the −35 element within the cbbL promoter was changed to the consensus E. coli nucleotide sequence (TTGACA). This promoter was shown to have high activity in E. coli (Jeffke, T. et al., J. Bacteria 181, 4374-4380 (1999)). The modified promoter was then amplified using primers flanked by NcoI and SacI restriction sites, and the amplicon was cloned into p72, resulting in plasmid p91.

TABLE 1
Plasmids and strains used in this study.

| Plasmid or strain | Relevant characteristics | Reference or source |
|---|---|---|
| Plasmids | | |
| pUC19 | general cloning vector | New England |
| | | Biolabs |
| #3716 | derivative of pLO11, contains the pBAD promoter | Oliver Lenz, Berlin, |
| | and araC gene | Germany |
| p62 | #3716 derivative with pUC19 multiple cloning site | This work |
| p72 | p62 derivative with NcoI restriction site in araC gene | This work |
| p91 | p72 derivative with modified R. eutropha cbbL | This work |
| | promoter | |
| p91BuOH(1) | synthetic butanol operon (hbd, crt, ter, atoB, adhE2) | This work |
| p91BuOH(1)lptB | synthetic butanol operon with essential lptB gene | This work |
| Strains | | |
| BW25113 | wild-type strain | ATCC, Manassas, |
| | | Virginia |
| RKE01 | BW25113 ΔlptB p91BuOH(1)lptB | This work |
| RKE03 | BW25113 ΔlptB ΔadhE p91BuOH(1)/lptB | This work |
| RKE05 | BW25113 ΔlptB ΔadhE Δpta p91BuOH(1)lptB | This work |
| RKE07 | BW25113 ΔlptB ΔadhE Δpta ΔldhA | This work |
| | p91BuOH(1)lptB | |
| RKE09 | BW25113 ΔlptB ΔadhE Δpta ΔldhA ΔfrdABCD | This work |
| | p91BuOH(1)/lptB | |

The hbd and crt genes were amplified from C. acetobutylicum genomic DNA and cloned into plasmid pBBR1MCS-3. Specifically, the hbd gene was cloned into the KpnI and XhoI restriction sites, and the crt gene was cloned into the XhoI and SmaI restriction sites, resulting in plasmid pBBR1MCS-3HC. The ter gene was amplified from Treponema denticola genomic DNA and cloned into the SmaI and SpeI restriction sites of pBBR1MCS-3HC, resulting in plasmid pBBR1MCS-3HCT. Then the entire hbd, crt, and ter nucleotide region from pBBR1MCS-3HCT was amplified and cloned into the SacI and XbaI multiple cloning site of p91, resulting in plasmid p91HCT. Next, the atoB gene from E. coli was amplified from genomic DNA and cloned into the XbaI multiple cloning site of p91HCT, resulting in plasmid p91HCTatoB. The adhE2 gene was amplified from C. acetobutylicum genomic DNA and cloned into the XbaI and HindIII multiple cloning sites of p91HCTatoB, resulting in plasmid p91HCTatoBadhE2. This plasmid was designated as p91BuOH(1). Finally, the lptB gene was amplified from E. coli genomic DNA and cloned between the hbd and crt genes of p91BuOH(1), using an available XhoI restriction site. The resulting plasmid was designated as p91BuOH(1)lptB.

To construct the plasmid addicted 1-butanol producing strains, plasmid p91BuOH(1)lptB was first used to construct strain RKE01, an lptB chromosomal gene deletion strain carrying a previously described lptB allele (Sherman, D. J. et al., Proc. Natl. Acad. Sci. U.S.A. 111, 4982-4987 (2014)). The adhE gene was inactivated in E. coli strain RKE01, resulting in strain RKE03. Next, the pta gene was inactivated in strain RKE03, resulting in strain RKE05. Strain RKE07 was constructed by inactivation of the ldhA gene in strain RKE05. Finally, the frdABCD genes were inactivated in strain RKE07, resulting in strain RKE09.

All amplicons and constructed plasmids were sequenced to ensure that no mutations occurred. In terms of a ribosome-binding site (RBS) for translation of each gene, the RBS within the cbbL promoter was used for the hbd gene and the remaining genes used an optimized RBS (Sichwart, S., Hetzler, S., Broker, D. and Steinbuchel, A., Appl. Environ. Microbiol. 77, 1325-1334 (2011)). All chromosomal deletion alleles were transferred using Plvir transduction by selecting for the kanamycin-resistance cassette that was used to replace the deleted gene of interest (Silhavy, T. J., Berman, M. L. and Enquist, L. W. in Experiments with gene fusions. (Cold Spring Harbor Laboratory, Cold Spring Harbor, N.Y., 1984) and confirmed by genomic polymerase chain reaction sequence analysis. After successful transduction of each deletion allele, the kanamycin cassette was excised as previously described (Cherepanov, P. P. and Wackernagel, W., Gene 158, 9-14 (1995)).

Culture conditions for 1-butanol production. Single colonies were seed cultured in 10 ml Terrific Broth (TB; 12 g tryptone, 24 g yeast extract, 2.31 g KH₂ PO₄, 12.54 g K₂ HPO₄, per liter of water, supplemented with glycerol or glucose) with 12.5 μg/ml tetracycline at 30° C. with 200 rpm shaking in a rotary incubator shaker overnight and inoculated at 5% of the total culture volume into vessels for testing different 1-butanol production conditions. Four different growth conditions were examined; aerobic, anaerobic, semi-aerobic, and high cell density. For the aerobic 1-butanol production condition, 150 ml of TB was used in 250 ml shake flasks. Flasks were incubated at 30° C. with 200 rpm shaking in a rotary incubator shaker. For anaerobic 1-butanol production, 50 ml of TB in 160 ml serum bottles were purged with N₂ for 5 min, closed with a thick butyl rubber stopper and sealed with an aluminum crimp. Cultures were incubated at 30° C. without shaking. For semi-aerobic 1-butanol production, 5 ml of TB in a 12 ml capped test tubes were incubated at 30° C. with 200 rpm shaking. In addition, 50 ml of TB in 160 ml serum bottles were closed with a thick butyl rubber stopper and sealed with an aluminum crimp. These 50 ml semi-aerobic cultures were incubated at 30° C. with 200 rpm shaking in a rotary incubator shaker overnight and then moved to an incubator without shaking. For high cell density 1-butanol production conditions, 150 ml TB in 250 ml shake flasks were used. Flasks were incubated at 30° C. with 200 rpm shaking in a rotary incubator shaker overnight. The next morning, the cells were collected by centrifugation at 10,000 g for 10 min at 4° C. The cell pellet was then resuspended in 25 ml TB. The cell suspension was transferred to 160 ml serum bottles (purged with N₂ for 5 min for high density anaerobic growth, closed with a thick butyl rubber stopper, sealed with an aluminum crimp, and then autoclaved). Cultures were incubated at 30° C. without shaking.

To determine the relative stability and duration of 1-butanol production of semi-continuous aerobic batch cultures, 250 ml shake flasks containing 150 ml TB supplemented with 2% glycerol was initially used. Batch cultures were incubated at 30° C. with 200 rpm shaking in a rotary incubator shaker. This first batch culture was inoculated with 7.5 ml of an overnight 30 ml seed culture and cultured for 72 h. Then 135 ml of culture from the first batch culture was removed and replaced with 135 ml of fresh medium, resulting in the second batch culture. Likewise, the second batch culture was used to create the third batch culture. 1-butanol was measured at the end of each batch cycle (72 h) since this time frame resulted in maximal butanol production under the conditions described. Thus, the stability of the described PAS system was monitored over 210 h.

Unless otherwise noted, the culture pH was adjusted to around 7 using 5 N NaOH on a daily basis for all cultures. Samples were taken daily to monitor cell growth (OD₆₀₀), substrate (glycerol or glucose) consumption, and production of 1-butanol, butyric acid, ethanol, and acetic acid during the fermentation.

Detection of 1-butanol, glycerol, and glucose. The quantification of 1-butanol was performed independently in two different laboratories, each using a GC-2014 (Shimadzu, Columbia, Md.) gas chromatograph (GC) equipped with a flame ionization detector and a Stabilwax DA (Restek, Bellefonte, Pa.) column (30 m, 0.32 mm ID, 0.25 μm film thickness). For the first method, the GC oven temperature was initially held at 50° C., raised with a gradient of 10° C./min until a temperature of 125° C. was reached, then raised with a gradient of 20° C./min until a temperature of 220° C. was reached; this was held for 10 min. Helium was used as the carrier gas. One μl of spent medium was injected in a split ratio of 1:5. For the second method, the GC was operated at an injection temperature of 200° C. with 1 μl of sample injected with an auto injector (AOC-20i, Shimadzu). The column temperature was initially held at 80° C. for 3 min, then increased at a constant rate of 30° C. per min to 150° C., and held at 150° C. for 3.7 min. Both methods yielded identical measurements of 1-butanol levels in the culture media. The second method was also used to quantify ethanol, acetate, and butyrate. Fermentation broth samples were analyzed for their glycerol and glucose consumption using a high performance liquid chromatograph (HPLC) equipped with an organic acid analysis column (Bio-Rad HPX-87H) and a refractive index detector (Shimadzu RID-10A) at 45° C., with 0.007 M H₂ SO₄ as the eluent at 0.6 ml/min.

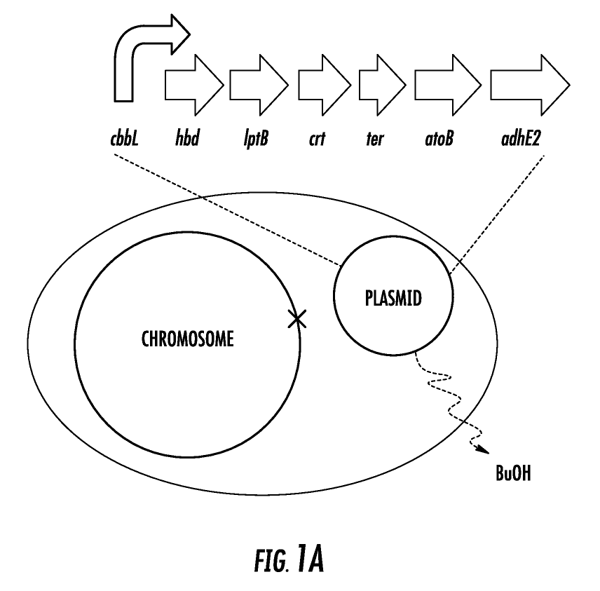

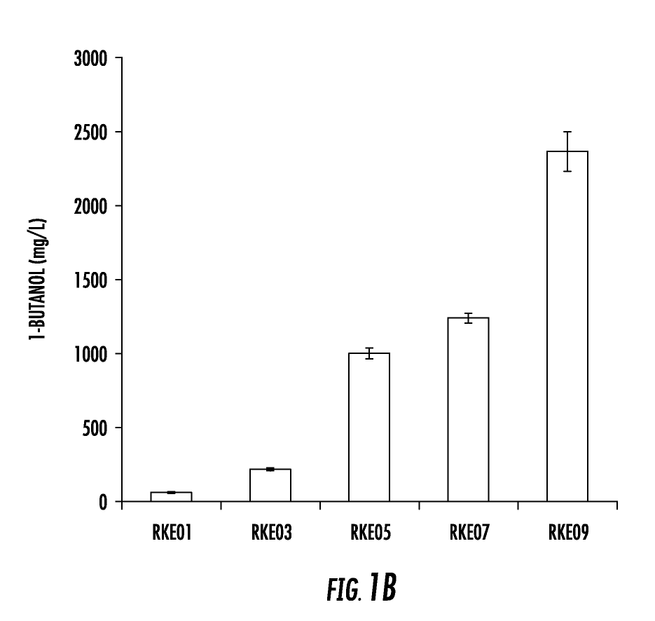

Results. With the rationale provided in FIG. 1A, plasmids p91BuOH(1) and p91BuOH(1)lptB were separately transformed into E. coli strain BW25113; deletion of the chromosomal lptB gene was performed via Plvir phage transduction. As expected, the strain that contained plasmid p91BuOH(1) was not viable due to the absence of either a chromosomal or plasmid-based essential lptB gene. Plasmid p91BuOH(1)lptB, however, rescued growth in the chromosomal lptB gene deletion mutant strain due to expression of the plasmid-based essential lptB gene, thus resulting in the construction of strain RKE01. The selection of genes for the synthetic 1-butanol operon used in this study was based on prior knowledge. For instance, it had been previously demonstrated that clostridial and non-clostridial gene products are capable of the multistep conversion of acetyl-CoA to 1-butanol (Shen, C. R. et al., Appl. Environ. Microbiol. 77, 2905-2915 (2011)); Atsumi, S. et al., Metab. Eng. 10, 305-311 (2008)). Therefore, specific 1-butanol genes that catalyze this conversion to construct a synthetic 1-butanol operon were acquired. Strain RKE01, which is addicted to plasmid p91BuOH(1)lptB, contains all the required genes for 1-butanol production (FIG. 1A).

Strain RKE01 was grown in small-scale cultures using 5 ml TB medium in 12 ml screw capped test tubes supplemented with 2% glycerol under semi-aerobic conditions and then examined for the production of 1-butanol, in the absence of co-inducer and antibiotic. Strain RKE01 produced 56 mg/L of 1-butanol (FIG. 1B). Previous studies, however, had indicated that increased 1-butanol production occurred after inactivation of the synthesis of fermentative products such as ethanol, acetate, lactate, and succinate (Shen, C. R. et al., Appl. Environ. Microbiol. 77, 2905-2915 (2011)). Therefore, the orderly and additive deletion of genes encoding enzymes of these fermentative pathways was performed using RKE01 as a parent strain. This resulted in the construction of strains RKE03(ΔadhE), RKE05(ΔadhEΔpta), RKE07(ΔadhEΔptaΔldhA), and RKE09(ΔadhEΔptaΔldhAΔfrdABCD). Inactivation of the alcohol dehydrogenase gene (adhE) in strain RKE03 increased 1-butanol production by 3.8-fold (FIG. 1B). In strain RKE05, the additional inactivation of the phosphotransacetylase gene (pta) resulted in an additional 4.3-fold increase in 1-butanol production (FIG. 1B). Next, a further 1.2-fold increase in 1-butanol production was obtained when the lactate dehydrogenase gene (ldhA) was additionally inactivated in strain RKE07 (FIG. 1B). Lastly, when the fumarate reductase genes (frdABCD) were also inactivated, in strain RKE09, a level of 2.3 g/L of 1-butanol was produced (FIG. 1B). In summary, the step-wise inactivation of the indicated genes resulted in a 40-fold increase in butanol production in strain RKE09 as compared to strain RKE01.

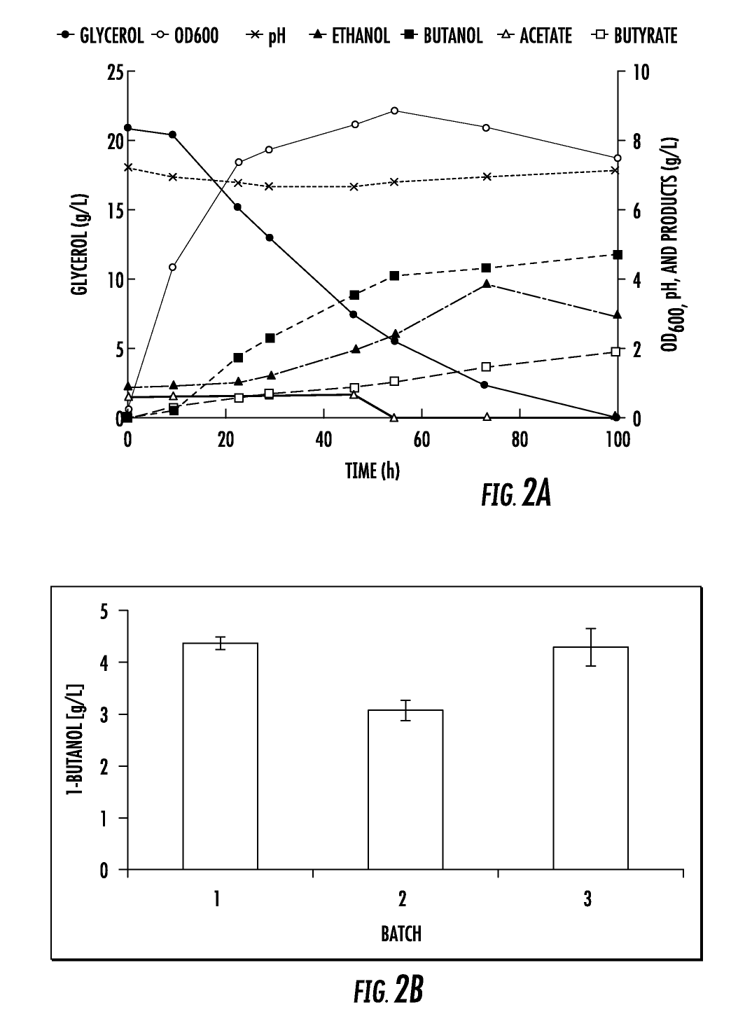

Strain RKE09 was further examined for 1-butanol production in larger-scale cultures in TB medium under different growth conditions. Aerobic, anaerobic, semi-aerobic, and high cell density growth conditions were examined with the supplement of 2% glycerol. 1-butanol production increased to 4.7 g/L (yield: 0.23 g butanol/g of glycerol used, productivity: 0.047 g 1-butanol synthesized/L/h) under aerobic growth conditions but decreased somewhat under anaerobic, semi-aerobic, and high cell density growth conditions (Table 2A and FIG. 2A). The maximum level of 1-butanol production (4.7 g/L) compared favorably to the previously mentioned initial small-scale semi-aerobic production studies (2.3 g/L). Aerobic growth was further examined by increasing glycerol levels from 2% to 5%, however, no significant increase in 1-butanol production occurred. Anaerobic, semi-aerobic, and high cell density 1-butanol production was also reexamined, by using 2% glucose as opposed to 2% glycerol. The production of 1-butanol increased with 2% glucose supplementation, as compared to 2% glycerol (Table 2A). Similarly, when glucose was increased to 5%, 1-butanol production was further improved under these conditions (Table 2A).

TABLE 2A
Large-scale 1-butanol production in strain RKE09 under different growth
conditions with media supplemented with either glycerol or glucose.

| | 1-butanol (titer: g/L, yield: g/g, productivity: g/L/h)^{a} | | |
|---|---|---|---|
| | 2% glycerol | 2% glucose | 5% glucose |
| Aerobic^{b} | 4.7, 0.23, 0.047 | ND^{c} | 3.3, 0.084, 0.036 |
| Anaerobic^{d} | 1.1, 1.02, 0.0035 | 2.6, 0.14, 0.028 | 4.1, 0.15, 0.044 |
| Semi- | 1.4, 0.30, 0.015 | 2.2, 0.28, 0.028 | 3.6, 0.26, 0.038 |
| aerobic^{e} | | | |
| High cell | 1.6, 0.042, 0.006 | 2.1, 0.38, 0.0088 | 3.4, 0.20, 0.029 |
| density^{f} | | | |

^{a}titer, g butanol produced/L; yield, g butanol produced/g substrate utilized; productivity, g butanol produced/L/h
^{b}150 ml culture in a 250 ml non-sealed flask.
^{c}not determined
^{d}50 ml culture in a N₂ purged 160 ml sealed serum bottle.
^{e}50 ml culture in a 160 ml sealed serum bottle.
^{f}25 ml culture (resuspended from 150 ml culture) in a N₂ purged 160 ml sealed serum bottle.

In larger-scale experiments using varying growth parameters, the requirement for the addition of tetracycline was examined to determine whether maximal 1-butanol production was affected by its presence or absence; e.g. to ensure that plasmid-borne genes were maintained in strain RKE09. While it was demonstrated that strain RKE09 produced 1-butanol under small-scale test tube growth conditions without antibiotic addition, it was important to verify that production levels were maintained in larger cultures. Therefore, experiments were repeated and tested under aerobic, anaerobic, semi-aerobic, and high cell density growth conditions with larger scale cultures, using media supplemented with 5% glucose. Cultures were grown in the presence or absence of antibiotic (tetracycline). The production of 1-butanol was monitored in these cultures and it was clear that 1-butanol levels in strain RKE09 were similar whether the antibiotic was present or absent (Table 2B).

TABLE 2B
1-butanol production in strain RKE09 in the presence or absence of antibiotic.

| | 1-butanol (titer: g/L, yield: g/g, productivity: g/L/h) | | | |
|---|---|---|---|---|
| Antibiotic^{a} | Aerobic^{b} | Anaerobic^{c} | Semi-aerobic^{d} | High cell density^{e} |
| + | 3.3, 0.084, 0.036 | 4.1, 0.15, 0.044 | 3.6, 0.26, 0.038 | 3.4, 0.20, 0.029 |
| − | 3.1, 0.084, 0.033 | 4.4, 0.14, 0.039 | 4.4, 0.26, 0.020 | 4.3, 0.23, 0.045 |

^{a}Tetracycline at 12.5 μg/ml was used where indicated.
^{b}150 ml culture in a 250 ml non-sealed flask.
^{c}50 ml culture in a N₂ purged 160 ml sealed serum bottle.
^{d}50 ml culture in a 160 ml sealed serum bottle.
^{e}25 ml culture (resuspended from 150 ml culture) in a N₂ purged 160 ml sealed serum bottle.

An experiment was devised whereby strain RKE09 was cultured until it maximally produced 1-butanol in the absence of co-inducers and antibiotics. After 72 h, cells from this initial culture were used to inoculate fresh media in a second culture, which was allowed to grow for 72 h as before. Cells from this second culture were then used to inoculate a third culture and maintained for another 72 h. This successive 216 h experiment indicated that there was no instability or substantial decrease in 1-butanol production over this time (FIG. 2B). Thus, the plasmid was stably maintained over this time without the addition of antibiotics, and the promoter was also actively maintained, resulting in stable gene expression over the time of this experiment.

Conclusion. Our results demonstrate that modification of the initial PAS strain increased 1-butanol production from 56 mg/L to 2.3 g/L. Further studies were performed under larger-scale growth conditions and a maximum titer of 4.7 g/L was produced under aerobic growth with 2% glycerol. Under anaerobic conditions, the 1-butanol titer was highest with glucose supplementation as opposed to glycerol addition, with a titer of 4.1 g/L of 1-butanol (yield: 0.15 g butanol/g of glucose utilized, and the productivity was at 0.044 g butanol produced/L/h). In addition, strain RKE09 was able to stably produce high levels of 1-butanol over a time period of over 200 hours during semi-continuous growth with no signs of instability. The PAS described here is versatile, being independent of carbon source or growth mode, as compared to other systems. Finally, the PAS-based 1-butanol production strain can be applied to industrial-scale, low-cost production, particularly with additional strain modifications. It was previously demonstrated, for example, that over-expression of genes of the formate dehydrogenase (Shen, C. R. et al., Appl. Environ. Microbiol. 77, 2905-2915 (2011)) and the pyruvate dehydrogenase complex (Bond-Watts, B. B., Bellerose, R. J. & Chang, M. C., Nat. Chem. Biol. 7, 222-227 (2011)) provide increased levels of NADH and markedly improved 1-butanol production in E coli ((Bond-Watts, B. B., Bellerose, R. J. & Chang, M. C., Nat. Chem. Biol. 7, 222-227 (2011)). Based on these experiments, it is expected that a similarly modified strain such as RKE09 can continuously produce high levels of 1-butanol, without the need for co-inducers or antibiotics.

The major finding from this study is that plasmid-addicted strains containing constitutive promoters can be highly useful for synthetic biology approaches for low-cost industrial synthesis of bioproducts and biofuels.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A plasmid addiction system comprising:
a host cell comprising one or more inactivated host cell essential genes; and
a plasmid comprising one or more plasmid essential genes operably linked to a constitutively active promoter.

2. The plasmid addiction system of claim 1, wherein the one or more inactivated host cell essential genes negatively effects and/or imbalances the redox balance of the host cell.

3. The plasmid addiction system of claim 2, wherein expression of the one or more plasmid essential genes in the host cell restores the redox balance of the host cell.

4. The plasmid addiction system of claim 1, wherein the host cell is a gram negative bacteria.

5. The plasmid addiction system of claim 4, wherein the bacteria is Escherichia coli, Ralstonia eutropha, Rhodobacter, Rhodopseudomonas, or Rhodospirillum.

6. The plasmid addiction system of claim 1, wherein the inactivated host cell essential genes encode one or more genes associated with lipopolysaccharide biogenesis.

7. The plasmid addiction system of claim 1, wherein the plasmid essential genes encode one or more genes associated with fermentation.

8. The plasmid addiction system of claim 1, where in the plasmid essential genes rescue and/or compensate for the inactivated host cell essential genes.

9. The plasmid addiction system of claim 7, wherein the genes associated with fermentation are selected from the group alcohol dehydrogenase, lactate dehydrogenase and/or fumarate reductase.

10. The plasmid addiction system of claim 1, wherein the promoter is from Ralstonia eutropha.

11. The plasmid addiction system of claim 10, wherein the promoter is cbbL.

12. The plasmid addiction system of claim 1, wherein the host cell of the plasmid addiction system is capable of growing under anaerobic growth conditions.

13. The plasmid addiction system of claim 1, wherein the one or more plasmid essential genes comprises a synthetic 1-butanol operon.

14. The plasmid addiction system of claim 1, wherein the inactivated host cell essential gene is lptB and the plasmid essential gene comprises lptB and a synthetic 1-butanol operon.

15. The plasmid addiction system system of claim 1, wherein the plasmid addiction system produces 1-butanol.

16. The plasmid addiction system system of claim 15, wherein the one or more plasmid essential genes comprises one or more genes associated with 1-butanol pathway.

17. A method of producing a recombinant cell, the method comprising:
(a) contacting a host cell comprising one or more inactivated host cell essential genes with a plasmid comprising one or more plasmid essential genes operably linked to a constitutively active promoter;
(b) selecting the recombinant cell comprising said plasmid.

18. The method of claim 17, wherein the method further comprises culturing the recombinant cell in anaerobic conditions in the absence of a co-inducer and/or antibiotic.

19. The method of claim 17, wherein the one or more inactivated host cell essential genes imbalances the redox balance of the host cell.

20. The method of claim 17, wherein the one or more plasmid essential genes restores the redox balance of the host cell.

21-33. (canceled)
